(19) United States
(12) Patent Application Publication
(10) Pub. No.: US 2007/0078175 A1
(43) Pub. Date: Apr. 5, 2007

(54) Administration of novel phenylfurylmethylthiazolidine-2,4-dione and phenylthienylmethylthiazolidine-2,4-dione compounds for stimulating or inducing the growth of keratinous fibers and/or slowing loss thereof

(75) Inventors: Christophe Boulle, Paris (FR); Maria Dalko, Gif S/Yvette (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 11/543,193

(22) Filed: Oct. 5, 2006

Related U.S. Application Data
(60) Provisional application No. 60/726,207, filed on Oct. 14, 2005.

(30) Foreign Application Priority Data
Oct. 5, 2005 (FR) 05/53017

(51) Int. Cl. A61K 31/427; C07D 417/02; A61K 8/49

(52) U.S. Cl. 514/369; 548/183

(57) ABSTRACT

Novel phenylfurylmethylthiazolidine-2,4-dione and phenylthienylmethylthiazolidine-2,4-dione compounds having the following formula and the salts or their solvates thereof: 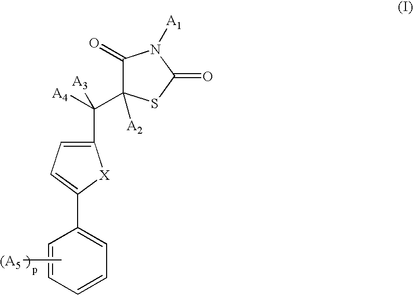 are useful for stimulating or inducing the growth of keratinous fibers and/or slowing down their loss and/or increasing their density and/or improving their appearance, and also for caring for or making up keratinous fibers, e.g., for caring for or making up the hair or eyelashes.

### CROSS-REFERENCE TO COMPANION APPLICATION

Copending U.S. Patent Application No. ______ [Attorney Docket No. 1016800-000784], filed concurrently herewith and assigned to the assignee hereof.

### CROSS-REFERENCE TO PRIORITY/PROVISIONAL APPLICATIONS

This application claims priority under 35 U.S.C. § 119 of FR 05/53017, filed Oct. 5, 2005, and of U.S. Provisional Application No. 60/726,207, filed Oct. 14, 2005, each hereby expressly incorporated by reference and each assigned to the assignee hereof.

### BACKGROUND OF THE INVENTION

1. Technical Field of the Invention

The present invention relates to novel phenylfurylmethylthiazolidine-2,4-dione and phenylthienylmethylthiazolidine-2,4-dione compounds and administration thereof for stimulating or inducing the growth of keratinous fibers and/or slowing down their loss and/or increasing their density and/or improving their appearance.

This invention also relates to compositions for caring for or making up keratinous fibers, comprising an effective amount of a phenylfurylmethylthiazolidine-2,4-dione or phenylthienylmethylthiazolidine-2,4-dione compound having specific formulae, which compositions are useful for increasing the density of these fibers and/or for improving their appearance.

More especially, the present invention relates to compositions for caring for or making up the hair or eyelashes which are useful to induce and/or stimulate their growth and/or to slow down their loss and/or to increase their density and/or to improve their appearance.

The invention additionally relates to a cosmetic treatment process, whether regime or regimen, useful to stimulate the growth of keratinous fibers and in particular of the hair and/or slow down their loss.

The human keratinous fibers to which the present invention relates are in particular the hair, eyebrows, eyelashes, beard hair or moustache hair. More especially, this invention relates to human hair and/or eyelashes.

In particular, the present invention relates to compositions for caring for and/or making up the hair or eyelashes, for topical application, comprising a phenylfurylmethylthiazolidine-2,4-dione or phenylthienylmethylthiazolidine-2,4-dione compound having a specific formula, which are useful to increase their density and/or enhance their appearance.

2. Description of Background and/or Related and/or Prior Art

The growth of the hair and its renewal are mainly determined by the activity of the hair follicles and of their matrix environment. Their activity is cyclical and essentially comprises three phases, namely, the anagen phase, the catagen phase and the telogen phase.

The anagen phase (active or growth phase), which lasts several years and during which the hair lengthens, is succeeded by a very short and transitory catagen phase, which lasts a few weeks. During this phase, the individual hair undergoes a change, the follicle atrophies and its implantation in the skin appears less and less deep.

The terminal phase or telogen phase, which lasts several months, corresponds to a resting phase of the follicle and the individual hair finishes by falling out. At the end of this resting period, a new follicle is regenerated there and another cycle recommences.

The hair is therefore continuously renewed and, of the approximately 150,000 individual hairs which make up the hair, approximately 10% are at rest and will be replaced in a few months.

The natural loss of the hair can be estimated, on average, at a few hundred hairs per day for a normal physiological state. This constant physical renewal process undergoes a natural change during the course of aging; the hairs become finer and their cycles shorter.

In addition, various causes can result in a significant, temporary or definitive, hair loss. The hair can be lost or detrimentally affected during recovery from pregnancy (post partum), during conditions of undernourishment or of dietary imbalances, during physiological stress, or during conditions of asthenia or of hormonal dysfunctioning, as may be the case during the course of or during recovery from the menopause. Hair can also be lost or detrimentally affected in connection with seasonal phenomena.

It may also be a matter of alopecia, which is essentially due to a disturbance of hair renewal which results, first, in an acceleration in the frequency of the cycles to the detriment of the quality of the hair and then of its amount. The successive growth cycles result in hair which is increasingly fine and increasingly short, which is gradually converted to an unpigmented down. Areas are preferentially affected, in particular the temples or the front of the head in men, and, in women, a diffuse alopecia of the vertex is observed.

The term “alopecia” also covers a whole family of conditions of the hair follicle having, as a final consequence, partial or general permanent hair loss. It is a matter more particularly of androgenic alopecia. In a significant number of cases, early hair loss takes place in genetically predisposed subjects; it is then a matter of androchronogenetic alopecia. This form of alopecia affects men in particular.

In some dermatosis conditions of the scalp with an inflammatory nature, such as, for example, psoriasis or seborrhoeic dermatitis, hair loss can be greatly increased or can result in highly disrupted cycles of the follicles.

There has been a search for many years, in the cosmetic or pharmaceutical industry, for compositions which make it possible to eliminate or reduce alopecia and in particular to induce or stimulate hair growth or to decrease hair loss.

From this viewpoint, a large number of compositions comprising very diverse active principles, such as, for example, 2,4-diamino-6-piperidinopyrimidine 3-oxide or “minoxidil”, disclosed in U.S. Pat. Nos. 4,139,619 and 4,596,812, or its numerous derivatives, such as those disclosed in EP-0,353 123, EP-0,356 271, EP-0,408 442, EP-0-522,964, EP-0-420,707, EP-0-459,890 and EP-0-519,819, have already been proposed.

Clinical studies have demonstrated that PGF_{2α} analogues have the property of bringing about the growth of body hairs and eyelashes in man and animals (Murray A. and Johnstone M. D., 1997, Am. J. Opht., 124(4), 544-547). In man, tests carried out on the scalp have shown that a prostaglandin E₂ analogue (viprostol) has the property of increasing hair density (Roenigk H. H., 1988, Clinic Dermatol., 6(4), 119-121).

Furthermore, WO 98/33497 discloses pharmaceutical compositions comprising prostaglandins or prostaglandin derivatives intended to combat hair loss in man. Prostaglandins of the A₂, F_{2α} and E₂ type are mentioned as preferred.

However, prostaglandins are molecules with a very short biological half-life which act autocrinally or paracrinally, this reflecting the local and labile nature of the metabolism of prostaglandins (Narumiya S. et al., 1999, Physiol. Rev., 79(4), 1193-1226).

It thus appears important, to maintain and/or increase hair density in man, to retain the endogenous reserves of PGF_{2α} and of PGE₂ in the various compartments of the hair follicle or of its immediate cutaneous environment.

A solution which gives good results is the administration of compounds which are inhibitors of lipoxygenase and/or inducers of cyclooxygenase for the purpose of promoting hair growth; one hypothesis is that the administration of such compounds directs the metabolism of the fatty acids towards the endogenous synthesis of prostaglandins in preference to other routes.

However, to further improve such results, it would be desirable to be able to prolong the activity of the prostaglandins involved in the growth and the preservation of the individual living hair.

Furthermore, it is well known that the programs of differentiation of the keratinocytes of the epidermis and of the hair follicle are clearly different. Thus, it is known that the keratins of the hair shaft represent a family (Langbein et al., 2001, J. Biol. Chem., 276, 35123-35132) distinct from that expressed in the epidermis, that differentiation markers such as keratins K₁ and K₁₀ are not expressed in the hair follicle and in particular in the outer sheath (Lenoir et al., 1988, Dev. Biol., 130, 610-620), that trichohyalin (O'Guin et al., 1992, J. Invest. Dermatol., 98, 24-32) and keratin K6irs (Porter et al., 2001, Br. J. Dermatol., 145, 558-568) are expressed in the hair follicle, in particular in the inner sheath, but not in the epidermis, and that cyclooxygenase type 1, while it is expressed in the epidermis, is not expressed in the keratinocytes of the hair follicle but in the dermal papilla (Michelet et al., 1997, J. Invest. Dermatol., 108, 205-209).

It has now been demonstrated by the assignee hereof that an enzyme specifically involved in the decomposition of these prostaglandins is present in the dermal papilla of the individual hair, which is a determining compartment for the life of the individual hair. This is because the assignee hereof has now proved the presence of type-1 15-hydroxyprostaglandin dehydrogenase (abbreviated to 15-PGDH) therein. In addition, it has shown that the inhibition of type-1 15-PGDH has a beneficial effect on hair growth.

This is why the present invention relates to hair care or treatment compositions comprising at least one specific inhibitor of type-1 15-hydroxyprostaglandin dehydrogenase and a physiologically acceptable medium.

Type-1 15-PGDH is a key enzyme in the deactivation of prostaglandins, in particular of PGF_{2α} and of PGE₂, which are important mediators of the growth and survival of the individual hair. It corresponds to the EC 1.1.1.141 classification and is NAD⁺-dependent. It has been isolated from pig kidney; its inhibition by a thyroid hormone, triiodothyronine, at doses much greater than physiological doses has in particular been observed. For its part, type-2 15-PGDH is NADP-dependent.

Phenylfuran and phenylthiophene compounds, which may or may not be in salt form, having an inhibitory activity with regard to type-1 15-hydroxyprostaglandin dehydrogenase are known from WO 04/028441. However, the assignee hereof has found that these compounds exhibit the disadvantage of being colored and thus have a tendency, on application, to color keratinous substances, in particular keratinous fibers of the hair, eyebrows, eyelashes, beard hair or moustache hair.

### SUMMARY OF THE INVENTION

It has now been found that certain heterocyclic compounds and in particular certain phenylfurylmethylthiazolidine-2,4-dione and phenylthienylmethylthiazolidine-2,4-dione compounds, which may or may not be in the salt form, have, surprisingly, a favorable activity in improving the density of human keratinous fibers, in particular hair fibers. Furthermore, it has also been found that these compounds are inhibitors of 15-hydroxyprostaglandin dehydrogenase. These compounds do not exhibit the disadvantages of the phenylfuran or phenylthiophene compounds of the prior art.

The present invention thus features novel phenylfurylmethylthiazolidine-2,4-dione and phenylthienylmethylthiazolidine-2,4-dione compounds of formula (I), which will be defined in detail below, and their salts and/or their solvates.

The present invention also features the administration of an effective amount of at least one phenylfurylmethylthiazolidine-2,4-dione or phenylthienylmethylthiazolidine-2,4-dione compound of formula (I) and their salts and/or their solvates as bioactive agents for inducing and/or stimulating the growth of keratinous fibers, in particular human keratinous fibers, and/or slowing down their loss and/or increasing their density.

The phenylfurylmethylthiazolidine-2,4-dione or phenylthienylmethylthiazolidine-2,4-dione compounds and their salts and/or their solvates in accordance with the invention have the following structural formula: 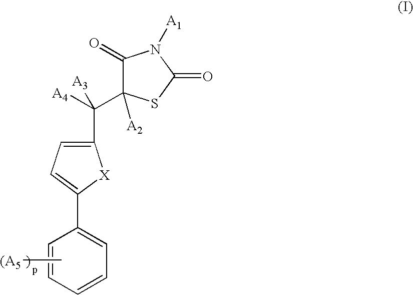
in which:
a) X is O or S;
b) A₁ is:
   1) a hydrogen atom;
   2) a linear or branched and saturated or unsaturated C₁-C₂₀ alkyl radical optionally substituted by one or more Z₁ groups;
c) the A₂, A₃, A₄ and A₅ radicals, which may be identical or different, are each:
   1) a hydrogen atom;
   2) a linear or branched and saturated or unsaturated C₁-C₂₀ alkyl radical optionally substituted by one or more Z₁ radicals;
   3) a saturated or unsaturated hydrocarbon ring member having 3 to 7 atoms which is optionally substituted by one or more Z₁ groups;
   4) a Z₁ radical;
d) p ranges from 0 to 5 inclusive and, in the event that p≧1, the A₅ substituents can be identical or different;
e) Z₁ is:
   1) a halogen, such as F, Cl or Br;
   2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
   3) a linear or branched and saturated or unsaturated C₁-C₂₀ alkyl radical;
   4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
   1) a hydrogen atom;
   2) a linear or branched and saturated or unsaturated C₁-C₂₀ alkyl radical optionally substituted by one or more Z₃ groups;
   3) a saturated or unsaturated ring member having 4 to 15 atoms, optionally containing at least one heteroatom selected from among O, N or S, optionally fused to another ring, these rings optionally being substituted by one or more Z₃ groups; this ring is advantageously a phenyl;
g) the Z₃ radical is:
   1) a Z₄ group;
   2) a linear or branched and saturated or unsaturated C₁-C₂₀ alkyl radical optionally substituted by one or more Z₄ groups;
   3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
   1) a halogen, such as F, Cl or Br;
   2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
   1) a hydrogen atom;
   2) a linear or branched and saturated or unsaturated C₁-C₂₀ alkyl radical;
   3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

### DETAILED DESCRIPTION OF BEST MODE AND SPECIFIC/PREFERRED EMBODIMENTS OF THE INVENTION

The term “salts of compound of formula (I)” means, according to the invention, the organic or inorganic salts of a compound of formula (I).

Exemplary inorganic salts according to the invention are the sodium or potassium salts and the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogencarbonates, sulfates, hydrogenphosphates or phosphates.

Exemplary organic salts according to the invention are salts of triethanolamine, monoethanolamine, ethanolamine, hexadecylamine and N,N,N′,N′-tetrakis(2-hydroxypropyl)ethylenediamine and those of organic acids, such as citrates, lactates, glycolates, gluconates, acetates, propionates, fumarates, oxalates and tartrates.

Exemplary solvates of the compounds of formula (I) are the hydrates, alcohol solvates or water/alcohol solvates.

According to the invention, the compounds of formula (I) are in the isolated or discrete form, that is to say non-polymeric form.

The term “at least one” according to the invention means one or more (2, 3 or more). In particular, the composition can comprise one or more compounds of formula (I). This or these compounds can be in the tautomeric form. This or these compounds can be enantiomers and/or diastereoisomers or a mixture of these isomers, in particular a racemic mixture.

The term “alkyl radical” means, according to the invention, a hydrocarbon radical which can be linear or branched and saturated or unsaturated. In particular, the alkyl radical has from 1 to 20 carbon atoms, preferably from 1 to 10. Examples of alkyl radicals according to the invention are the methyl, ethyl, isopropyl, n-butyl, tert-butyl, n-hexyl, 2-ethylhexyl, ethylene and propylene radicals.

Exemplary halogen atoms according to the invention are chlorine, fluorine and bromine atoms and preferably the chlorine and fluorine atoms.

Exemplary saturated ring members according to the invention are the cyclopropyl, cyclobutyl, cyclopentyl, cyclohexyl, cycloheptyl and adamantyl radicals.

Exemplary unsaturated ring members are the cyclohexenyl and phenyl radicals. Exemplary fused hydrocarbon rings of the invention are naphthyl and azulenyl radicals.

Exemplary fused rings of different nature according to the invention are the benzofuran, dibenzofuran, benzothiophene, benzothiazole, indole, benzimidazole, quinoline, isoquinoline, quinazoline, carboline, chromene, carbazole and fluorene radicals.

Exemplary ring members containing a carbonyl or thiocarbonyl functional group according to the invention are the following rings:

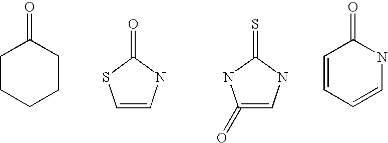

Exemplary heterocycles according to the invention are the azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine and crown ether 15-C-5 rings. Preferred are the pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine and morpholine ring members.

The preferred compounds of formula (I) in accordance with the invention are those for which the following conditions are satisfied:
(a) the A₁, A₂, A₃ and A₄ radicals simultaneously are hydrogen;
(b) p has the value 0, 1 or 2;
(c) the A₅ radical or radicals, which may be identical or different, are each a saturated or unsaturated and linear or branched C₁-C₁₀ alkyl radical optionally substituted by a Z₁ group or a Z₂ group;
(d) Z₁ is a halogen, CN, NO₂, CF₃, OZ₂, OCOZ₂, COOZ₂ or a phenyl ring optionally fused to another ring and optionally substituted by one or more Z₄ groups as defined above;

(e) Z₂ is hydrogen or a saturated or unsaturated and linear or branched C₁-C₁₀ alkyl radical.

Exemplary preferred compounds of formula (I) are the following compounds and their salts and/or their solvates:

| Compound | Name |
|---|---|
| 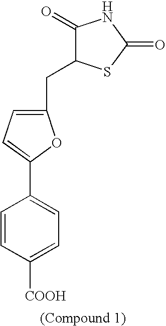 | 4-{5-[(2,4-Dioxo-1,3- thiazolidin-5-yl)methyl]-2- furyl}benzoic acid |
| 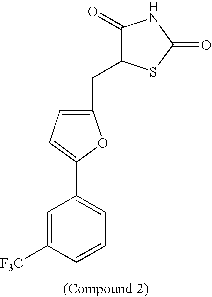 | 5-([5-[3- (Trifluoromethyl)phenyl]-2- furyl]methyl)-1,3-thiazolidine- 2,4-dione |
| 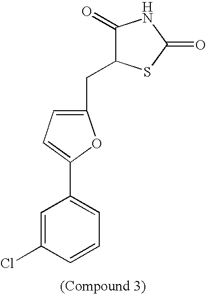 | 5-{[5-(3-Chlorophenyl)-2- furyl]methyl}-1,3-thiazolidine- 2,4-dione |
| 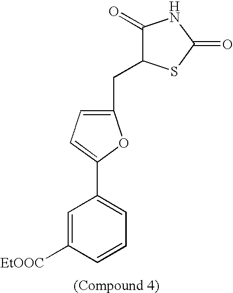 | Ethyl 3-{5-[(2,4-dioxo-1,3- thiazolidin-5-yl)methyl]-2- furyl}benzoate |
| 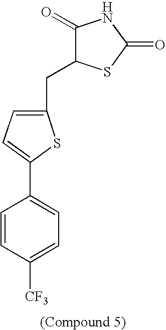 | 5-({5-[4- (Trifluoromethyl)phenyl]thien-2- yl}methyl)-1,3-thiazolidine-2,4- dione |
| 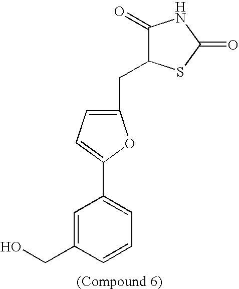 | 5-([5-[3- (Hydroxymethyl)phenyl]-2- furyl]methyl)-1,3-thiazolidine- 2,4-dione |
| 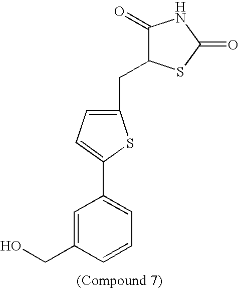 | 5-({5-[3- (Hydroxymethyl)phenyl]-2- thienyl}methyl)-1,3-thiazolidine- 2,4-dione |
| 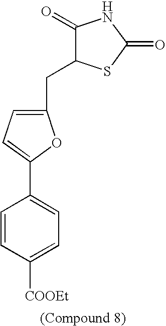 | Ethyl 4-{5-[(2,4-dioxo-1,3- thiazolidin-5-yl)methyl]-2- furyl}benzoate |
| 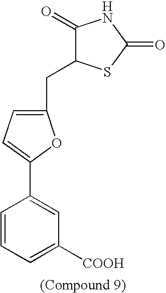 | 3-{5-[(2,4-Dioxo-1,3- thiazolidin-5-yl)methyl]-2- furyl}benzoic acid |
| 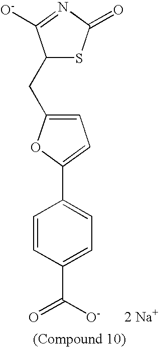 | Disodium salt of 4-{5-[(2,4- dioxo-1,3-thiazolidin-5- yl)methyl]-2-furyl}benzoic acid |
| 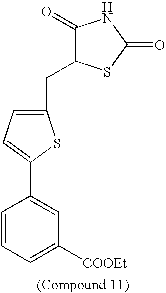 | Ethyl 3-{5-[(2,4-dioxo-1,3- thiazolidin-5-yl)methyl]-2- thienyl}benzoate |
| 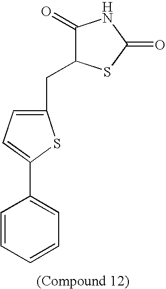 | 5-[(5-Phenyl-2-thienyl)methyl]- 1,3-thiazolidine-2,4-dione |
| 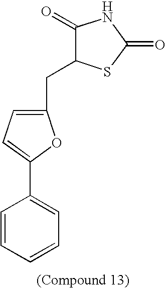 | 5-[(5-Phenyl-2-furyl)methyl]- 1,3-thiazolidine-2,4-dione |

More particularly preferred among these compounds are those selected from among:

4-{5-[(2,4-dioxo-1,3-thiazolidin-5-yl)methyl]-2-furyl}benzoic acid of formula:

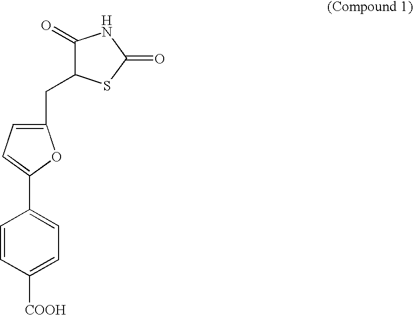

disodium salt of 4-{5-[(2,4-dioxo-1,3-thiazolidin-5-yl)methyl]-2-furyl}benzoic acid of formula: 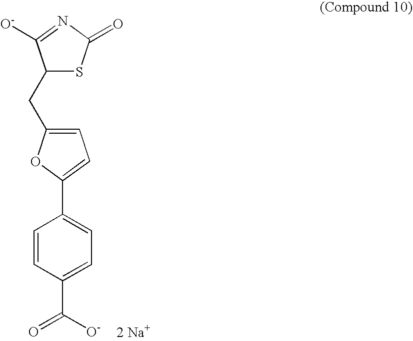
and their salts and/or their solvates.

The compounds of formula (I) in accordance with the invention can be prepared by a process for the reduction of a compound corresponding to the following structural formula: 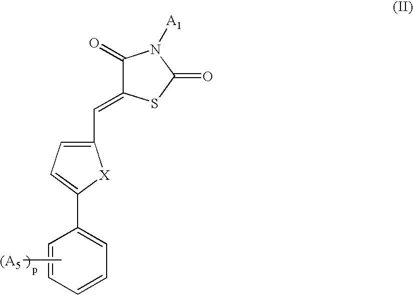
in which A₁ and A₅ and X and p have the same definitions as in the formula (I) defined above, and according to the following reaction scheme:

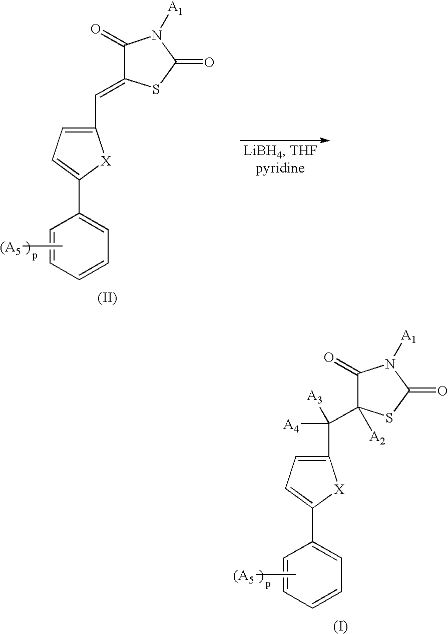

The compounds of formula (II) are disclosed, as is the synthesis thereof, in WO 04/028441.

The present invention also features the administration, in particular cosmetic application, of at least one phenylfurylmethylthiazolidine-2,4-dione or phenylthienylmethylthiazolidine-2,4-dione compound of formula (I) or of one of its salts and/or of its solvates as defined above as bioactive agent for inducing and/or stimulating the growth of keratinous fibers, in particular human keratinous fibers, and/or slowing down their loss and/or increasing their density.

The present invention also features the cosmetic formulation of at least one phenylfurylmethylthiazolidine-2,4-dione or phenylthienylmethylthiazolidine-2,4-dione compound of formula (I) or of one of its salts and/or of its solvates into compositions for caring for or treating keratinous fibers, in particular human keratinous fibers, in order to reduce their loss and/or increase their density. In addition, such compositions make it possible to maintain the keratinous fibers in good condition and/or make possible an improvement in their appearance.

This invention also features the use of at least one phenylfurylmethylthiazolidine-2,4-dione or phenylthienylmethylthiazolidine-2,4-dione compound of formula (I) or of one of its salts and/or of its solvates in the manufacture of compositions for caring for or treating keratinous fibers, in particular human keratinous fibers, which compositions are useful to induce and/or stimulate the growth of the said fibers and/or slow down their loss and/or increase their density.

The present invention also relates to the keratinous fibers of mammals of the animal type (dogs, horses or cats, for example).

The human keratinous fibers to which this invention relates are in particular the hair, eyebrows, eyelashes, beard hair or moustache hair.

Consequently, the invention also features the cosmetic application of at least one phenylfurylmethylthiazolidine-2,4-dione or phenylthienylmethylthiazolidine-2,4-dione compound of formula (I) or of one of its salts and/or of its solvates in cosmetic compositions for human hair care for reducing hair loss and/or increasing its density and/or treating androchronogenetic alopecia.

The present invention also features the use of at least one phenylfurylmethylthiazolidine-2,4-dione or phenylthienylmethylthiazolidine-2,4-dione compound of formula (I) or of one of its salts and/or of its solvates in the preparation of hair compositions for man which are useful to induce and/or stimulate hair growth and/or to slow down hair loss and/or to increase hair density and/or to treat androgenic alopecia.

The present invention also features the formulation of at least one phenylfurylmethylthiazolidine-2,4-dione or phenylthienylmethylthiazolidine-2,4-dione compound of formula (I) or of one of its salts and/or of its solvates into cosmetic compositions for human hair care in order to treat alopecia of natural origin and in particular androgenic origin.

This invention also features formulation of at least one phenylfurylmethylthiazolidine-2,4-dione or phenylthienylmethylthiazolidine-2,4-dione compound of formula (I) or of one of its salts and/or of its solvates into compositions for human hair care which are useful to treat alopecia of natural origin and in particular androgenic origin. Thus, these compositions make it possible to maintain the hair in good condition and/or to combat natural hair loss and more especially that of men.

This invention also features formulating at least one phenylfurylmethylthiazolidine-2,4-dione or phenylthienylmethylthiazolidine-2,4-dione compound of formula (I) or of one of its salts and/or of its solvates into cosmetic compositions for caring for and/or making up human eyelashes, in order to induce and/or stimulate the growth of the eyelashes and/or to increase their density.

The present invention also features formulating at least one phenylfurylmethylthiazolidine-2,4-dione or phenylthienylmethylthiazolidine-2,4-dione compound of formula (I) or of one of its salts and/or of its solvates into compositions for caring for or treating human eyelashes, which compositions are useful to induce and/or stimulate the growth of the eyelashes and/or to increase their density. These compositions thus make it possible to maintain the eyelashes in good condition and/or to improve their condition and/or their appearance.

The present invention also features administration of at least one phenylfurylmethylthiazolidine-2,4-dione or phenylthienylmethylthiazolidine-2,4-dione compound of formula (I) or of one of its salts and/or of its solvates as type-1 15-hydroxyprostaglandin dehydrogenase inhibitors, in particular for the human skin.

This invention also features the use of at least one phenylfurylmethylthiazolidine-2,4-dione or phenylthienylmethylthiazolidine-2,4-dione compound of formula (I) or of one of its salts and/or of its solvates in the manufacture of compositions useful to treat disorders related to the presence of type-1 15-hydroxyprostaglandin dehydrogenase, in particular in man.

The present invention also features a process for the cosmetic treatment of human keratinous fibers (in particular hair or eyelashes) and/or of the skin from where the said fibers emerge, including the scalp and eyelids, whether regime or regimen, comprising topically applying, onto the said keratinous fibers and/or the said skin, a cosmetic composition comprising at least one phenylfurylmethylthiazolidine-2,4-dione or phenylthienylmethylthiazolidine-2,4-dione compound of formula (I) or one of its salts and/or of its solvates, in maintaining this composition in contact with the keratinous fibers and/or the skin from where the said fibers emerge and optionally in rinsing the said fibers and/or the said skin.

This treatment process clearly exhibits the characteristics of a cosmetic process in so far as it makes it possible to improve the attractiveness of human keratinous fibers and in particular of the hair and eyelashes by giving them greater vigor and an improved appearance. In addition, it can be used daily for several months without a medical prescription.

More especially, the present invention features a process for the cosmetic care of human hair and/or the human scalp for the purpose of improving their condition and/or their appearance, comprising topically applying, onto the hair and/or the scalp, a cosmetic composition comprising an effective amount of at least one phenylfurylmethylthiazolidine-2,4-dione or phenylthienylmethylthiazolidine-2,4-dione compound of formula (I) or of one of its salts and/or of its solvates, in maintaining this composition in contact with the hair and/or the scalp and optionally in rinsing the hair and/or the scalp.

This invention also features a process for the cosmetic care of and/or for making up human eyelashes for the purpose of improving their condition and/or their appearance, comprising topically applying, onto the eyelashes and/or eyelids, a mascara composition comprising at least one phenylfurylmethylthiazolidine-2,4-dione or phenylthienylmethylthiazolidine-2,4-dione compound of formula (I) or one of its salts and/or of its solvates and in maintaining this composition in contact with the eyelashes and/or eyelids. This mascara composition can be applied alone or as an undercoat of a conventional pigmented mascara and can be removed like a conventional pigmented mascara.

The present invention also features a care and/or mascara compositions for the hair or eyelashes, comprising a cosmetically acceptable medium for topical application and a phenylfurylmethylthiazolidine-2,4-dione or phenylthienylmethylthiazolidine-2,4-dione compound of formula (I) or one of its salts and/or of its solvates.

This invention also features compositions for caring for or making up keratinous fibers comprising, in a physiologically acceptable medium, in particular a cosmetic medium, at least one phenylfurylmethylthiazolidine-2,4-dione or phenylthienylmethylthiazolidine-2,4-dione compound or one of its salts and/or of its solvates and at least one additional active principle which promotes the regrowth of human keratinous fibers and/or which limits their loss, selected from among aminexil, FP receptor agonists, prostaglandins and their derivatives, and vasodilators and selected more especially from aminexil, minoxidil, latanoprost, (5E)-7-{(1R,2R,3R,5S)-3,5-dihydroxy-2-[(3R)-3-hydroxy-5-phenylpentyl]cyclopentyl}hept-5-enoic acid, butaprost and travoprost.

The present invention also features the cosmetic administration of a phenylfurylmethylthiazolidine-2,4-dione or phenylthienylmethylthiazolidine-2,4-dione compound of formula (I) or of one of its salts and/or of its solvates as bioactive agent for preserving the amount and/or the activity of prostaglandins in the hair follicle.

This invention also features the use of at least one phenylfurylmethylthiazolidine-2,4-dione or phenylthienylmethylthiazolidine-2,4-dione compound of formula (I) or of one of its salts and/or of its solvates in the manufacture of compositions useful to preserve the amount and/or the activity of prostaglandins in the hair follicle.

The term “15-hydroxyprostaglandin dehydrogenase inhibitor” means a compound of formula (I) which is capable of inhibiting or reducing the activity of the enzyme type-1 15-PGDH, in particular in man, and/or is capable of inhibiting, reducing or slowing down the reaction catalyzed by this enzyme.

According to an advantageous embodiment of the invention, the compound of formula (I) is a specific inhibitor of 15-PGDH; the term “specific inhibitor” means an active principle which is not or only to a slight extent an inhibitor of the synthesis of prostaglandins, in particular of the synthesis of PGF_{2α} or PGE₂. According to a specific embodiment of the invention, the inhibitor of 15-PGDH is not or only to a slight extent an inhibitor of the synthesis of prostaglandins, in particular of the synthesis of PGF_{2α} or PGE₂. In particular, the inhibitor of type-1 15-PGDH is not or only to a slight extent an inhibitor of prostaglandin synthase (referred to as PGF synthase or PGFS).

This is because it has now been found that PGF synthase is also expressed in the dermal papilla. The maintenance of an effective amount of prostaglandins at the site of action thus results from a complex biological equilibrium from the synthesis and the decomposition of these molecules. The exogenous contribution of compounds which inhibit catabolism will therefore be less effective if this activity is combined with inhibition of the synthesis of these prostaglandins.

The compounds of formula (I), which may or may not be salified and which may or may not be solvated, advantageously exhibit an inhibitory activity for 15-PGDH which is greater than the activity inhibiting PGF synthase. These compounds are referred to as selective inhibitors of 15-PGDH with respect to PGF synthase. In particular, the ratio of the inhibitory activity for PGF synthase to the inhibitory activity for 15-PGDH for a given concentration, which activities are determined in particular by the concentration which inhibits 50% of the enzymatic activity of PGF synthase (IC_{50fs}) with respect to the concentration which inhibits 50% of the enzymatic activity of 15-PGDH (IC_{50dh}), is at least greater than 1 and in particular at least 3:1, advantageously greater than or equal to 5:1.

Hereinafter, and unless expressly indicated, the use of the term “compound of formula (I)” should be understood as meaning both the compound of formula (I) and one of its salts or of its solvates, in particular hydrates, or one of its solvated salts (in particular hydrated salts).

The effective amount of a compound of formula (I) (salified or non-salified, solvated or non-solvated) corresponds to the amount necessary to elicit the desired result (namely, to increase the density of keratinous fibers and in particular of the hair and eyelashes or to promote their growth). One skilled in the art is therefore in a position to evaluate this effective amount, which depends on the nature of the compound used, on the person to which it is applied and on the time of this application.

Also hereinafter, unless otherwise indicated, the amounts of the various ingredients of the composition are given as percentage by weight with respect to the total weight of the composition.

To provide an order of magnitude according to the invention, the compound of formula (I) (salified or non-salified, solvated or non-solvated) or a mixture of compounds of formula (I) and/or of their salts can be used in an amount representing from 10⁻³% to 10% of the total weight of the composition and preferably in an amount representing from 10⁻³% to 5% to better still from 10⁻²% to 2% of the total weight of the composition, for example from 0.5 to 2%.

The compositions of the invention can be for cosmetic or pharmaceutical applications. Preferably, the composition of the invention is for cosmetic application. Consequently, the composition should comprise a physiologically acceptable medium which is non-toxic and which is capable of being applied to human skin, including the scalp and eyelids, and to human keratinous fibers. The term “cosmetic” means, within the scope of the invention, a composition with a pleasant appearance, smell and feel.

The compounds of formula (I), which may or may not be salified and which may or may not be solvated, can be formulated into a composition which has to be ingested, injected or applied to the skin or to keratinous fibers (over any cutaneous region or all of the fibers to be treated).

According to the invention, the compound of formula (I) or a mixture of compounds of formula (I) can be administered orally in an amount of 0.1 to 300 mg per day, in particular of 5 to 10 mg/day.

A preferred composition of the invention is a composition for cosmetic use and in particular for topical application to the skin and keratinous fibers and more especially to the scalp, hair and eyelashes.

This composition can be provided in any known dosage form which is suited to the method of use.

For topical application to the skin or keratinous fibers, the composition can have the form of an aqueous, alcoholic or aqueous/alcoholic solution or suspension or of an oily suspension or solution, of an emulsion or dispersion with a more or less fluid consistency and in particular a liquid or semi-liquid consistency, obtained by dispersion of a fatty phase in an aqueous phase (O/W) or vice versa (W/O), of an (O/W) or (W/O) solid emulsion or dispersion, of an aqueous, aqueous/alcoholic or oily gel which is more or less fluid or solid, of a free or compact powder to be used as is or to be incorporated in a physiologically acceptable medium, or also of microcapsules or microparticles, or of vesicular dispersions of ionic and/or nonionic type.

Also intended are compositions in the form of a foam or in the form of a spray or aerosol then comprising a pressurized propellant.

They can thus be provided in the form of a lotion, serum, milk, O/W or W/O cream, gel, ointment, pomade, powder, balm, patch, impregnated pad, cake or foam.

In particular, the composition for application to the scalp or hair can be provided in the form of a hair care lotion, for example for daily or twice-weekly application, of a shampoo or of a hair conditioner, in particular for twice-weekly or weekly application, of a liquid or solid soap for cleaning the scalp, for daily application, of a product for shaping the hairstyle (lacquer, hairsetting product, styling gel), of a treatment mask, of a cream or of a foaming gel for cleaning the hair. They can also be provided in the form of a hair dye or mascara to be applied with a brush or comb.

Furthermore, for application to the eyelashes or body hairs, the compositions to which the invention relates can be provided in the form of a pigmented or non-pigmented mascara, to be applied with a brush to the eyelashes or, alternatively to the beard or moustache hairs.

For a composition for administration by injection, the composition can be provided in the form of an aqueous lotion or of an oily suspension. For administration by the oral route, the composition can be provided in the form of capsules, of granules, of syrups to be taken orally or of tablets.

According to a specific embodiment, the composition according to the invention is provided in the form of a hair cream or lotion, of a shampoo, of a hair conditioner, of a hair mascara or of a mascara for the eyelashes.

The amounts of the various constituents of the composition according to the invention are those generally employed in the fields under consideration. In addition, these compositions are formulated according to conventional methods.

When the composition is an emulsion, the proportion of the fatty phase can range from 2% to 80% by weight and preferably from 5% to 50% by weight with respect to the total weight of the composition. The aqueous phase is adjusted according to the content of fatty phase and of compound(s) (I) and according to the content of possible additional ingredients, in order to obtain 100% by weight. In practice, the aqueous phase is from 5% to 99.9% by weight.

The fatty phase can comprise fatty or oily compounds which are liquid at ambient temperature (25° C.) and atmospheric pressure (760 mmHg), generally known as oils. These oils may or may not be compatible with one another and may form a macroscopically homogeneous liquid fatty phase or a two- or three-phase system.

The fatty phase can, in addition to the oils, comprise waxes, gums, lipophilic polymers, or “pasty” or viscous products comprising solid parts and liquid parts.

The aqueous phase comprises water and optionally an ingredient miscible in any proportion with water, such as lower C₁ to C₈ alcohols, for example ethanol or isopropanol, polyols, such as propylene glycol, glycerol or sorbitol, or else acetone or ether.

The emulsifiers and coemulsifiers used to produce a composition in the form of an emulsion are those generally employed in the cosmetic and pharmaceutical fields. In addition, their nature depends on the sense of the emulsion. In practice, the emulsifier and optionally the coemulsifier are present in the composition in a proportion ranging from 0.1% to 30% by weight, preferably from 0.5% to 20% by weight and better still from 1% to 8%. In addition, the emulsion can comprise lipid vesicles and in particular liposomes.

When the composition is in the form of an oily solution or gel, the fatty phase can represent more than 90% of the total weight of the composition.

Advantageously, for a topical hair application, the composition is an aqueous, alcoholic or aqueous/alcoholic solution or suspension and better still a water/ethanol solution or suspension. The alcohol fraction can represent from 5% to 99.9% to better still from 8% to 80%.

For a topical mascara application, the composition of the invention is in particular in the form of a wax-in-water or wax-in-oil dispersion, of a gelled oil or of an aqueous gel, with or without pigment.

The compositions of the invention can comprise, in addition, other additional ingredients generally employed in the fields concerned selected from among solvents, thickeners or gelling agents for the aqueous phase or for the oily phase, coloring materials which are soluble in the medium of the composition, solid particles of the filler or pigment type, antioxidants, preservatives, fragrances, electrolytes, neutralizing agents, film-forming polymers, UV-blocking agents, such as sunscreens, cosmetic and pharmaceutical active principles with a beneficial effect on the skin or keratinous fibers, other than the compounds of formula (I), or their mixtures. These additives can be present in the composition according to the amounts generally employed in the cosmetic and dermatological fields and in particular in a proportion of 0.01 % to 50% of the total weight of the composition and better still of 0.1% to 20% to, for example, of 0.1% to 10%. These additives, depending on their nature, can be introduced into the fatty phase, into the aqueous phase and/or into the lipid vesicles and in particular liposomes.

Of course, one skilled in the art will take care to choose the possible additional ingredients and/or their amounts so that the advantageous properties of the composition according to the invention, namely the inhibition of type-1 15-PGDH and in particular the increase in the density of keratinous fibers, are not, or not substantially, detrimentally affected by the envisaged addition.

Exemplary solvents according to the invention include lower C₂ to C₈ alcohols, such as ethanol or isopropanol, propylene glycol and certain light cosmetic oils, such as C₆ to C₁₆ alkanes.

Exemplary oils according to the invention include oils of mineral origin (liquid petrolatum, hydrogenated isoparaffin), oils of vegetable origin (liquid fraction of shea butter, sunflower oil, apricot oil, fatty alcohol or fatty acid), oils of animal origin (perhydrosqualene), synthetic oils (fatty acid esters, purcellin oil), silicone oils (phenyltrimethicone, linear or cyclic polydimethylsiloxane) and fluorinated oils (perfluoropolyethers). Exemplary waxes, include silicone waxes, beeswax, rice wax, candelilla wax, carnauba wax, paraffin wax or polyethylene wax.

Exemplary emulsifiers according to the invention include glyceryl stearate or laurate, sorbitol stearates or oleates, alkyl dimethicone copolyols (with alkyl≧8) and their mixtures for a W/O emulsion. Also exemplary are polyethylene glycol monostearate or monolaurate, polyoxyethylenated sorbitol stearate or oleate, dimethicone copolyols and their mixtures for an O/W emulsion.

Exemplary hydrophilic gelling agents according to the invention include carboxyvinyl polymers (carbomer), acrylic copolymers, such as acrylate/alkylacrylate copolymers, polyacrylamides, polysaccharides, such as hydroxypropylcellulose, natural gums and clays and exemplary lipophilic gelling agents include modified clays, such as bentones, metal salts of fatty acids, such as aluminum stearates, hydrophobically treated silica, ethylcellulose or their mixtures.

Exemplary cosmetic or pharmaceutical active principles other than the compounds of formula (I) which can be used in the invention, include hydrophilic active principles, such as proteins or protein hydrolysates, amino acids, polyols, urea, allantoin, sugars and sugar derivatives, water-soluble vitamins, plant extracts (those of Iridaceae or of soya) and hydroxy acids, such as fruit acids or salicylic acid; and lipophilic active principles, such as retinol (vitamin A) and its derivatives, in particular esters (retinol palmitate), tocopherol (vitamin E) and its derivatives, in particular esters (tocopherol acetate), essential fatty acids, ceramides, essential oils, salicylic acid derivatives, such as 5-octanoylsalicylic acid, esters of hydroxy acids, phospholipids, such as lecithin, or their mixtures.

According to a specific embodiment of the invention, the compound of formula (I) or one of its salts and/or of its solvates can be combined with additional compounds which promote the regrowth and/or which limit the loss of keratinous fibers (hair, eyelashes). These additional compounds are selected in particular from among lipoxygenase inhibitors, such as disclosed in EP 648 488, bradykinin inhibitors, disclosed in particular in EP-845,700, prostaglandins and their derivatives, in particular those disclosed in WO 98/33497, WO 95/11003, JP 97-100091 or JP 96-134242, prostaglandin receptor agonists or antagonists, non-prostanoic prostaglandin analogues, such as disclosed in EP-1-175,891 and EP-1-175,890, WO 01/74307, WO 01/74313, WO 01/74314, WO 01/74315 or WO 01/72268, or their mixtures.

Other additional active compounds which promote the growth of keratinous fibers and/or which limit their loss (in particular the hair or eyelashes) which can be present in the composition according to the invention include vasodilators, anti-androgens, cyclosporins and their analogues, anti-microbials and anti-fungals, anti-inflammatories or retinoids, alone or as a mixture.

The vasodilators which can be used are in particular potassium channel agonists, including minoxidil and the compounds disclosed in U.S. Pat. Nos. 3,382,247, 5,756,092, 5,772,990, 5,760,043, 5,466,694, 5,438,058 or 4,973,474, cromakalim, nicorandil and diazoxide, alone or in combination.

The anti-androgens which can be used include in particular steroidal or non-steroidal inhibitors of 5α-reductase, such as finasteride and the compounds disclosed in U.S. Pat. No. 5,516,779, cyprosterone acetate, azelaic acid, its salts and its derivatives and the compounds disclosed in U.S. Pat. No. 5,480,913, flutamide, oxendolone, spironolactone, diethylstilbestrol and the compounds disclosed in U.S. Pat. Nos. 5,411,981, 5,565,467 and 4,910,226.

The anti-microbial or anti-fungal compounds can be selected from among selenium derivatives, octopirox, triclocarban, triclosan, zinc pyrithione, itraconazole, asiatic acid, hinokitiol, mipirocin, tetracyclines, in particular erythromycin and the compounds disclosed in EP-0-680,745, clinycin hydrochloride, benzoyl peroxide or benzyl peroxide, minocyclin and the compounds belonging to the class of the imidazoles, such as econazole, ketoconazole or miconazole or their salts, or nicotinic acid esters, including in particular tocopherol nicotinate, benzyl nicotinate and C₁-C₆ alkyl nicotinates, such as methyl nicotinate or hexyl nicotinate.

The anti-inflammatories can be selected from among steroidal anti-inflammatories, such as glucocorticoids or corticosteroids (for example: hydrocortisone), and non-steroidal anti-inflammatories, such as glycyrrhetinic acid and α-bisabolol, benzydamine, salicylic acid and the compounds disclosed in EP-0-770,399, WO 94/06434 and FR-2-268,523.

The retinoids can be selected from among isotretinoin, acitretin, tazarotene, retinal and adapalene.

Other additional active compounds for promoting the growth and/or limiting the loss of keratinous fibers, such as the hair and eyelashes, which can be used in combination with the compound of formula (I), which may or may not be salified and which may or may not be solvated, include aminexil, 6-O-[(9Z,12Z)-octadeca-9,12-dienoyl]hexapyranose, benzalkonium chloride, benzethonium chloride, phenol, oestradiol, chlorpheniramine maleate, chlorophyllin derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic acid or acylhexosaccharic acid, aryl-substituted ethylenes, N-acylated amino acids, flavonoids, ascomycin derivatives and analogues, histamine antagonists, saponins, proteoglycanase inhibitors, oestrogen agonists and antagonists, pseudopterins, cytokines and growth factor promoters, IL-1 or IL-6 inhibitors, IL-10 promoters, TNF inhibitors, benzophenones and hydantoin, retinoic acid; vitamins, such as vitamin D, analogues of vitamin B12 and pantothenol; triterpenes, such as ursolic acid and the compounds disclosed in U.S. Pat. Nos. 5,529,769, 5,468,888 or 5,631,282; anti-pruritic agents, such as thenaldine, trimeprazine or cyproheptadine; agents for combating parasites, in particular metronidazole, crotamiton or pyrethroids; calcium antagonist agents, such as cinnarizine, diltiazem, nimodipine, verapamil, alverine and nifedipine; hormones, such as oestriol or its analogues, thyroxine and its salts, or progesterone; FP receptor (receptor to prostaglandins of the F type) agonists, such as latanoprost, (5E)-7-{(1R,2R,3R,5S)-3,5-dihydroxy-2-[(3R)-3-hydroxy-5-phenylpentyl]cyclopentyl}hept-5-enoic acid, bimatoprost, travoprost, unoprostone and butaprost; their mixtures.

Advantageously, the compositions according to the invention comprise at least one 15-PGDH inhibitor as defined above and at least one prostaglandin or one prostaglandin derivative, such as, for example, prostaglandins of the 2 series, including in particular PGF_{2α} and PGE₂, in the salt or ester form (for example, the isopropyl esters), their derivatives, such as 16,16-dimethyl-PGE₂, 17-phenyl-PGE₂, 16,16-dimethyl-PGF_{2α} or 17-phenyl-PGF_{2α}, or prostaglandins of the 1 series, such as 11-deoxyprostaglandin E₁ or 1-deoxyprostaglandin E₁, in the salt or ester form, their analogues, in particular latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, butaprost, unoprostone, misoprostol, their salts or their esters.

Preferably, the compositions comprise at least one non-prostanoic agonist of the EP2 and/or EP4 receptors, in particular as disclosed in EP-1-175,892.

Also intended are compositions comprising at least the compound of formula (I), which may or may not be salified and which may or may not be solvated, in the liposomed form, such as disclosed in particular in WO 94/22468. Thus, the compound encapsulated in the liposomes can be delivered selectively to the hair follicle.

The compositions according to the invention can be topically applied to the areas of the scalp which are suffering from alopecia and to the hair of an individual and can optionally be maintained in contact for several hours and can optionally be rinsed out.

It is possible, for example, to apply the composition comprising an effective amount of a compound of formula (I), which may or may not be salified and which may or may not be solvated, in the evening, to maintain this composition in contact overnight and optionally to shampoo in the morning. These applications can be repeated daily for one or more months, depending on the individual.

Advantageously, in the regime or regimen according to the invention, from 5 and 500 μl of a solution or composition as defined above, comprising from 0.001% to 5% of 15-PGDH inhibitor, are applied to the areas of the scalp to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Synthesis of 5-[5-(3-(trifluoromethyl)phenyl)fur-2-ylmethyl]thiazolidine-2,4-dione (compound 2)

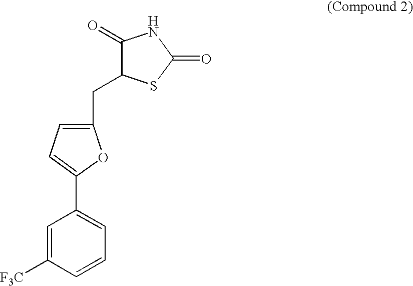 500 mg (1.47 mmol, 1 eq.) of 5-[5-(3-(trifluoromethyl)phenyl)fur-2-ylmethylene]thiazolidine-2,4-dione are dissolved in 2 ml of anhydrous THF in a three-necked flask under a stream of argon and 2 ml of pyridine over KOH are added. 1.71 ml of lithium borohydride are added very gently (effervescence and increase in temperature). The reaction medium is heated at 65° C. for 3 hours. The mixture is homogeneous. The medium is left at ambient temperature and then it is poured slowly onto a cooled 1N hydrochloric acid solution. The medium is then extracted twice with ethyl acetate. The combined organic phases are washed with water, dried with sodium sulfate, filtered and concentrated as much as possible.

The residue obtained is purified by chromatography on silica gel (eluant: dichloromethane). The expected compound is obtained in powder form.

Results:
Appearance: light beige powder
Weight=84 mg
Yield=17%
Melting point=138° C.
Molecular weight=341 g.mol⁻¹

Analyses:

The NMR and mass spectra are in accordance with the expected structure.

### Example 2

### Synthesis of 5-{[5-(3-chlorophenyl)-2-furyl]methyl}-1,3-thiazolidine-2,4-dione (compound 3)

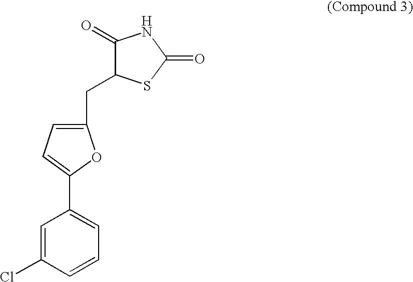

The procedure is identical to that of Example 1. The residue is purified by chromatography on silica gel (eluant: dichloromethane). The expected compound is obtained in powder form.

Results:
Appearance: powder
Weight=114 mg
Yield=6%
Melting point=104° C.
Molecular weight=307 g.mol⁻¹

Analyses:

The NMR and mass spectra are in accordance with the expected structure.

Elemental analyses:

| | | | | | |
|---|---|---|---|---|---|
| C | H | N | O | S | Cl |

Theory

| | | | | | |
|---|---|---|---|---|---|
| 54.60% | 3.30% | 4.60% | 15.60% | 10.40% | 11.50% |

Analysis

| | | | | | |
|---|---|---|---|---|---|
| 54.32% | 3.17% | 4.55% | 16.13% | 10.51% | 10.85% |

### Example 3

### Synthesis of 5-({5-[4-(trifluoromethyl)phenyl]thien-2-yl}methyl)-1,3-thiazolidine-2,4-dione (compound 5)

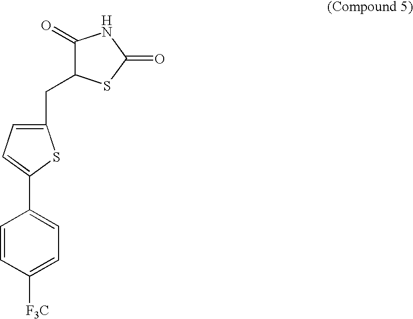

The procedure is identical to that of Example 1.

Purification is carried out on silica gel (eluant: heptane/ethyl acetate gradient: 80/20 to 50/50). The expected compound is obtained in powder form.

Results:
Appearance: light beige powder
Weight=79 mg
Yield=15%
Molecular weight=357 g.mol⁻¹

Analyses:

The NMR and mass spectra are in accordance with the expected structure.

### Example 4

### Synthesis of the compound 4-{5-[(2,4-dioxo-1,3-thiazolidin-5-yl)methyl]-2-furyl}benzoic acid (compound 1)

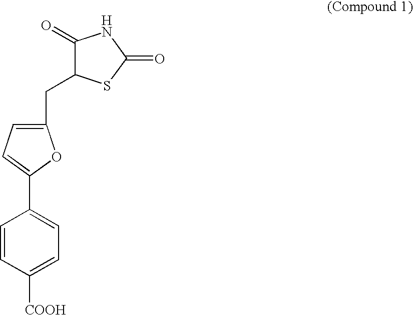

The procedure is identical to that of Example 1.

Analyses:

The NMR and mass spectra are in accordance with the expected structure.

### Example 5

### Synthesis of the compound disodium salt of 4-{5-[(2,4-dioxo-1,3-thiazolidin-5-yl)methyl]-2-furyl}benzoic acid (compound 10)

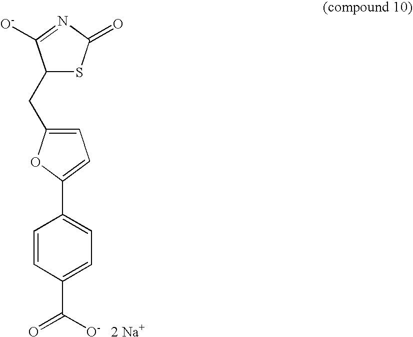 2.12 g of compound 1 are placed in 20 ml of water. 6.7 ml of a normal sodium hydroxide solution are added. The solid dissolves. After stirring for 30 minutes, the solution is poured onto acetone. A solid is formed, is filtered off and is then dried under vacuum. This solid, which comprises acetone, is dissolved in water and then brought to dryness. The residue is stirred in ethanol for 16 h, then filtered off and dried.
Appearance: powder
Weight=1.68 g
Yield=69%
Molecular weight=361 g.mol⁻¹

Analyses:

The NMR and mass spectra are in accordance with the expected structure.

### Example 6

### Demonstration of the Specific Inhibitory Properties of Compound 1 with Respect to 15-PGDH

Test on type-1 15-PGDH:

The enzyme 15-PGDH is obtained as disclosed in Application FR 02/05067, assigned to the assignee hereof, in suspension in a medium adjusted to a concentration of 0.3 mg/ml and then blocked at −80° C. For the requirements of the test, this suspension is defrosted and stored in ice.

Furthermore, a 100 mM, pH=7.4, Tris buffer comprising 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier) and 50 μM of prostaglandin E₂ (P4172, Sigma-Aldrich, L'isle D'Abeau Chesne, BP 701, 38297, Saint Quentin Fallavier) is prepared.

0.965 ml of this buffer (adjusted beforehand to 37° C.) is introduced into the cell of a spectrophotometer (Perkin-Elmer, Lambda 2) thermostatically controlled at 37° C., the measuring wavelength of which is adjusted to 340 nm. 0.035 ml of enzymatic suspension at 37° C. is introduced into the cell concomitantly with the recording (corresponding to an increase in the optical density at 340 nm). The maximum reaction rate is recorded.

The test values (comprising compound 1) are compared with the control value (without compound 1); the results shown represent either the percentage of inhibition of the enzymatic activity of 15-PGDH for a given concentration of compound of formula (I) or the concentration at which compound 1 reduces the enzymatic activity of 15-PGDH by 50%, namely IC_{50dh}.

The results are reported in the Table below.

| Compound | IC_{50dh} | |
|---|---|---|
| 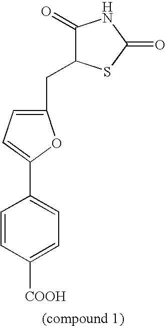 | 3.0 | μM |
| 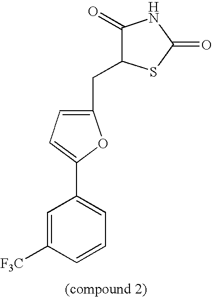 | 8.2 | μM |
| 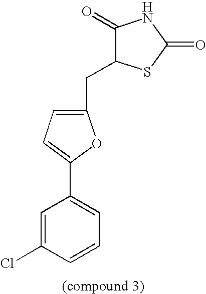 | 5.2 | μM |
| 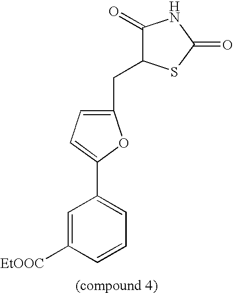 | 4.6 | μM |
| 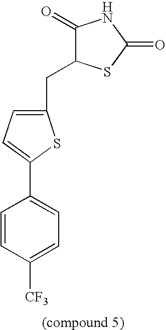 | 17.7 | μM |
| 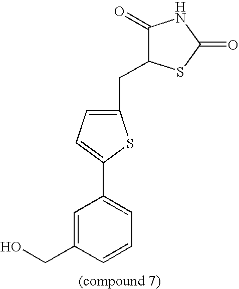 | 37.6 | μM |

It will thus be seen that compounds 1 to 4, 5 and 7 according to the invention are inhibitors of type-1 15-PGDH.

### Example 7

### Comparative Tests From a Compound of the Prior Art (WO 04/028441) and its Reduced Homologue According to the Invention

Hair Used:

The hair used for this experiment is of SA 40 grade (strong bleaching). Locks of hair weighing approximately 1 g are prepared and are washed with 0.4 g of shampoo. They are rinsed, dried with a hair dryer and then disentangled.

Protocol for Diluting the Products:

100 mg of product are weighed out and are treated with 3 ml of water, 1 ml of ethanol and 0.5 ml of a 20% aqueous ammonia solution (the medium being basic, the two compounds tested are thus in the salt form in the preparation medium). Stirring is maintained for 10 minutes. The lock is placed in a crystallizing dish without overlapping and then the solution is deposited over the lock using a pipette in order for the lock to be completely impregnated. The crystallizing dish is closed with a watchglass and then placed on a heating plate at 40° C. for 1 hour. The lock is subsequently superficially dried in absorbent paper and then disentangled and dried with a hair dryer.

Compounds Tested:

Ionic compound derived from 4-{5-[(2,4-disulfo-1,3-thiazolidin-5-ylidene)methyl]-2-furyl}benzoic acid (Z isomer) according to WO 04/028441 of formula:

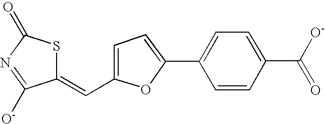

Compound according to the invention

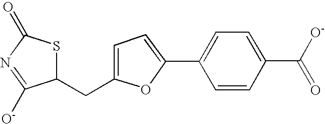

Control: the same operation is carried out without product.

It is found that the compound according to the prior art colors the locks of hair, in contrast to the compound according to the invention.

The compositions below are formulated by the usual techniques commonly employed in the cosmetic or pharmaceutical field.

### Example 8

Hair lotion:

| | | |
|---|---|---|
| Compound 1 | 1.00 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to reduce hair loss and to promote hair regrowth. It also makes it possible to improve the appearance of the hair.

### Example 9

Hair lotion:

| | | |
|---|---|---|
| Compound 1 | 1.00 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to reduce hair loss and to promote hair regrowth. It also makes it possible to improve the appearance of the hair.

### Example 10

Wax/water mascara:

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 1 | 1.00% |
| Black pigment | 5.00% |
| Preservative | q.s. |
| Water | q.s. for 100.00% |

This mascara is applied to the eyelashes like a conventional mascara with a mascara brush.

### Example 11

Hair lotion:

| | | |
|---|---|---|
| Compound 1 | 0.10 | g |
| Latanoprost | 0.10 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to reduce hair loss and to promote hair regrowth. It also makes it possible to improve the appearance of the hair.

### Example 12

Hair lotion:

### (5E)-7-{(1R,2R,3R,5S)-3,5-Dihydroxy-2-[(3R)-3-hydroxy-5-phenylpentyl]cyclopentyl}

| | | |
|---|---|---|
| hept-5-enoic acid | 0.10 | g |
| Compound 1 | 0.10 | g |
| Propylene glycol | 30.00 | g |
| Ethyl alcohol | 40.00 | g |
| Water | q.s. for 100.00 | g |

This lotion is applied to the scalp, once or twice daily, at the rate of 1 ml per application, the scalp being gently massaged in order to bring about the penetration of the active principle. The hair is subsequently dried in the open air. This lotion makes it possible to reduce hair loss and to promote hair regrowth. It also makes it possible to improve the appearance of the hair.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A phenylfurylmethylthiazolidine-2,4-dione or phenylthienylmethylthiazolidine-2,4-dione compound having the following structural formula or salt and/or solvate thereof: 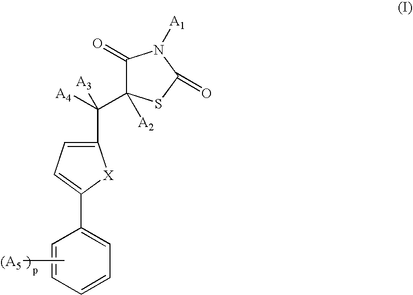
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ_{′2})OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

2. A compound as defined by claim 1 or salt and/or solvate thereof, wherein formula (I) the following conditions are satisfied:
(a) the A₁, A₂, A₃ and A₄ radicals simultaneously are each hydrogen;
(b) p has the value 0, 1 or 2;
(c) the A₅ radical or radicals, which may be identical or different, is (are each) a saturated or unsaturated and linear or branched C₁-C₁₀ alkyl optionally substituted by a Z₁ group or a Z₂ group;
(d) Z₁ is a halogen, CN, NO₂, CF₃, OZ₂, OCOZ₂, COOZ₂ or a phenyl ring optionally fused to another ring and optionally substituted by one or more Z₄ groups; and
(e) Z₂ is hydrogen or a saturated or unsaturated and linear or branched C₁-C₁₀ alkyl radical.

3. A compound as defined by claim 1, selected from the group consisting of the following compounds and/or their salts and/or their solvates:

| Compound | Name |
|---|---|
| 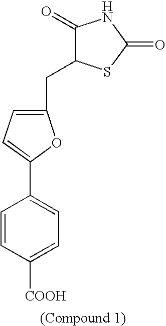 | 4-{5-[(2,4-Dioxo-1,3- thiazolidin-5-yl)methyl]-2- furyl}benzoic acid |
| 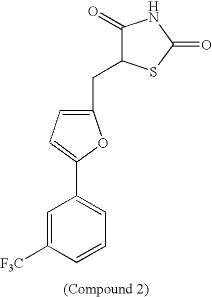 | 5-([5-[3- (Trifluoromethyl)phenyl]-2- furyl]methyl)-1,3-thiazolidine- 2,4-dione |
| 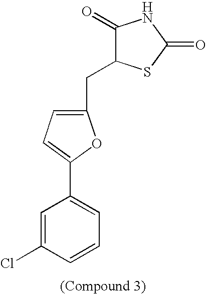 | 5-{[5-(3-Chlorophenyl)-2- furyl]methyl}-1,3-thiazolidine- 2,4-dione |
| 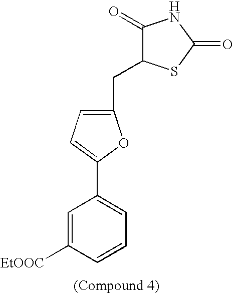 | Ethyl 3-{5-[(2,4-dioxo-1,3- thiazolidin-5-yl)methyl]-2- furyl}benzoate |
| 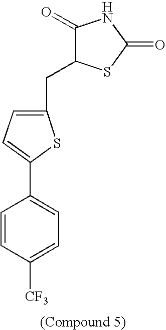 | 5-({5-[4- (Trifluoromethyl)phenyl]thien-2- yl}methyl)-1,3-thiazolidine-2,4- dione |
| 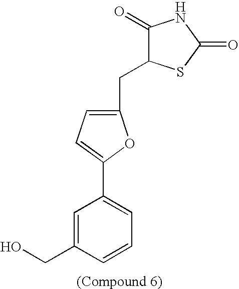 | 5-([5-[3- (Hydroxymethyl)phenyl]-2- furyl]methyl)-1,3-thiazolidine- 2,4-dione |
| 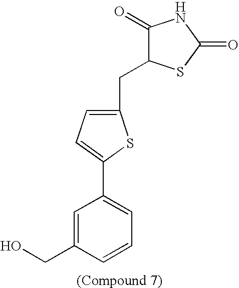 | 5-({5-[3- (Hydroxymethyl)phenyl]-2- thienyl}methyl)-1,3-thiazolidine- 2,4-dione |
| 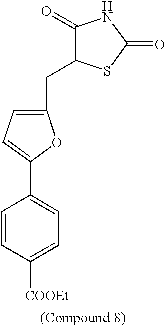 | Ethyl 4-{5-[(2,4-dioxo-1,3- thiazolidin-5-yl)methyl]-2- furyl}benzoate |
| 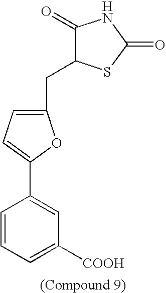 | 3-{5-[(2,4-Dioxo-1,3- thiazolidin-5-yl)methyl]-2- furyl}benzoic acid |
| 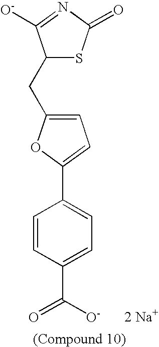 | Disodium salt of 4-{5-[(2,4- dioxo-1,3-thiazolidin-5- yl)methyl]-2-furyl}benzoic acid |
| 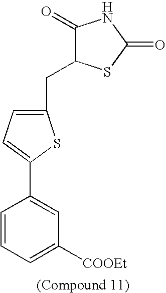 | Ethyl 3-{5-[(2,4-dioxo-1,3- thiazolidin-5-yl)methyl]-2- thienyl}benzoate |
| 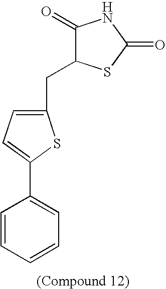 | 5-[(5-Phenyl-2-thienyl)methyl]- 1,3-thiazolidine-2,4-dione |
| 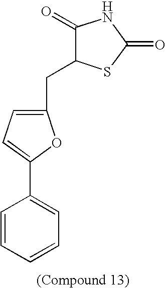 | 5-[(5-Phenyl-2-furyl)methyl]- 1,3-thiazolidine-2,4-dione |

4. A compound as defined by claim 1, selected from among the following compounds and/or their salts and/or their solvates:
4-{5-[(2,4-dioxo-1,3-thiazolidin-5-yl)methyl]-2-furyl}benzoic acid of formula: 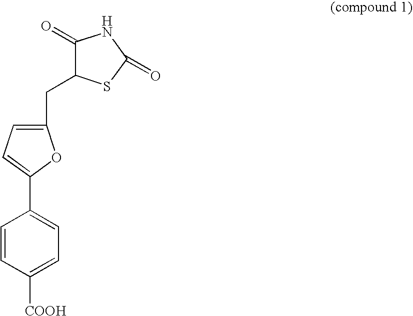
and disodium salt of 4-{5-[(2,4-dioxo-1,3-thiazolidin-5-yl)methyl]-2-furyl}benzoic acid of formula:

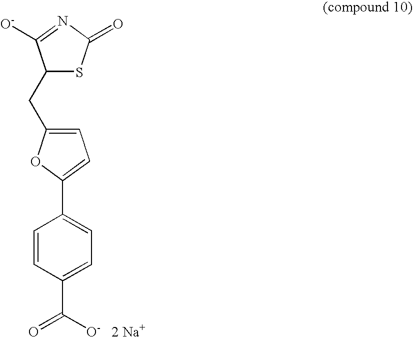

5. A regime or regimen for inducing and/or stimulating the growth of keratinous fibers and/or slowing down the loss and/or increasing the density thereof, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 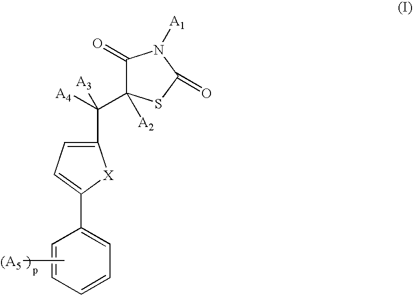
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

6. A regime or regimen for caring for and/or making up keratinous fibers of a subject in need of such treatment to reduce the loss and/or increase the density thereof, comprising topically applying thereon a thus effective amount of at least one phenylfurylmethylthiazolidine-2,4-dione or phenylthienylmethylthiazolidine-2,4-dione compound having the following structural formula (I) or salt and/or solvate thereof: 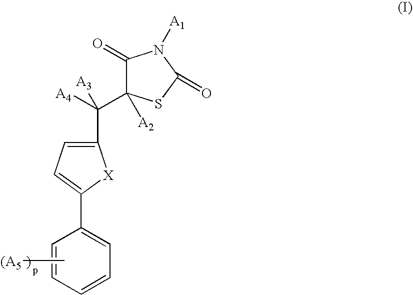
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

7. A regime or regimen for inhibiting human type-1 15-hydroxyprostaglandin dehydrogenase, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 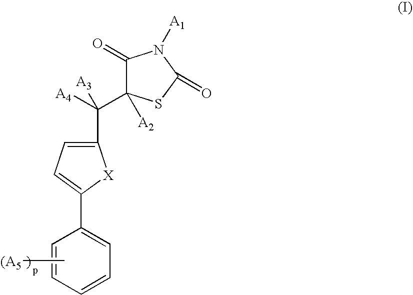
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

8. The regime or regime as defined by claim 5, said keratinous fibers comprising human hair, eyebrows, eyelashes, beard hair and/or moustache hair.

9. A regime or regimen for treating androchronogenetic alopecia and/or treating alopecia of natural origin, comprising administering to a subject in need of such treatment, for such period of time as required to elicit the desired response, a thus effective amount of at least one phenylfurylmethylthiazolidine-2,4-dione or phenylthienylmethylthiazolidine-2,4-dione compound having the following structural formula (I) or salt and/or solvate thereof: 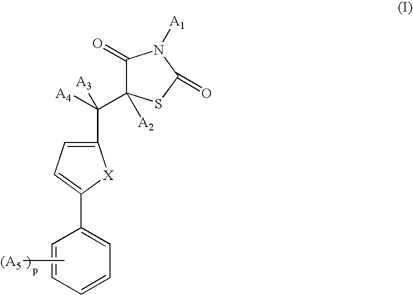
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

10. A cosmetic/pharmaceutical composition useful for the care and/or makeup of keratinous fibers and/or for inducing and/or stimulating the growth and/or slowing down the loss and/or increasing the density thereof, comprising a thus effective amount of at least one phenylfurylmethylthiazolidine-2,4-dione or phenylthienylmethylthiazolidine-2,4-dione compound having the following structural formula (I) or salt and/or solvate thereof: 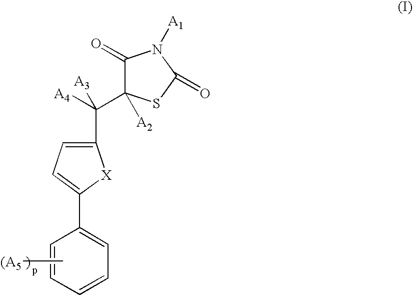
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals, formulated into a physiologically acceptable medium therefor.

11. The cosmetic/pharmaceutical composition as defined by claim 10, formulated for topical application.

12. The cosmetic/pharmaceutical composition as defined by claim 10, said at least one compound of formula (I) or salt and/or solvate thereof comprising from 10⁻³% to 10% by weight thereof.

13. The cosmetic/pharmaceutical composition as defined by claim 10, said at least one compound of formula (I) or salt and/or solvate thereof comprising from 10⁻²% to 10% by weight thereof.

14. The cosmetic/pharmaceutical composition as defined by claim 10, further comprising at least one other ingredient selected from the group consisting of solvents, thickeners or gelling agents, coloring materials which are soluble in the medium of the composition, fillers, pigments, antioxidants, preservatives, fragrances, electrolytes, neutralizing agents, film-forming polymers, UV-blocking agents, cosmetic and pharmaceutical active principles other than the compounds of formula (I), and mixtures thereof.

15. The cosmetic/pharmaceutical composition as defined by claim 14, further comprising at least one active principle selected from the group consisting of proteins or protein hydrolysates, amino acids, polyols, urea, allantoin, sugars and sugar derivatives, water-soluble vitamins, plant extracts, hydroxy acids, retinol and its derivatives, tocopherol and its derivatives, essential fatty acids, ceramides, essential oils, salicylic acid derivatives, esters of hydroxy acids, phospholipids and mixtures thereof.

16. The cosmetic/pharmaceutical composition as defined by claim 10, further comprising at least one additional active compound which promotes the regrowth and/or which limits the loss of keratinous fibers.

17. The cosmetic/pharmaceutical composition as defined by claim 16, further comprising at least one active compound selected from the group consisting of aminexil, 6-O-[(9Z,12Z)-octadeca-9,12-dienoyl]hexapyranose, lipoxygenase inhibitors, bradykinin inhibitors, prostaglandins and their derivatives, prostaglandin receptor agonists or antagonists, non-prostanoic prostaglandin analogues, vasodilators, anti-androgens, cyclosporins and their analogues, anti-microbials, anti-inflammatories, retinoids, benzalkonium chloride, benzethonium chloride, phenol, oestradiol, chlorpheniramine maleate, chlorophyllin derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic acid or acylhexosaccharic acid, aryl-substituted ethylenes, N-acylated amino acids, flavonoids, ascomycin derivatives and analogues, histamine antagonists, saponins, proteoglycanase inhibitors, oestrogen agonists and antagonists, pseudopterins, cytokines and growth factor promoters, IL-1 or IL-6 inhibitors, IL-10 promoters, TNF inhibitors, vitamins, benzophenones, hydantoin, retinoic acid, anti-pruritic agents, agents for combating parasites, anti-fungals, calcium antagonist agents, hormones, triterpenes, anti-androgen agents, steroidal or non-steroidal inhibitors of 5α-reductases, potassium agonists, FP receptor agonists, and mixtures thereof.

18. The cosmetic/pharmaceutical composition as defined by claim 17, comprising aminexil, FP receptor agonists, prostaglandins and their derivatives, and/or vasodilators.

19. The cosmetic/pharmaceutical composition as defined by claim 17, comprising aminexil, minoxidil, latanoprost, (5E)-7-{(1R,2R,3R,5S)-3,5-dihydroxy-2-[(3R)-3-hydroxy-5-phenylpentyl]cyclopentyl}hept-5-enoic acid, butaprost, bimatoprost and/or travoprost.

20. A regime or regimen for improving the condition and/or the appearance of human keratinous fibers and/or the skin from whence said fibers emerge, comprising topically applying thereon, a thus effective amount of at least one phenylfurylmethylthiazolidine-2,4-dione or phenylthienylmethylthiazolidine-2,4-dione compound having the following structural formula (I) or salt and/or solvate thereof: 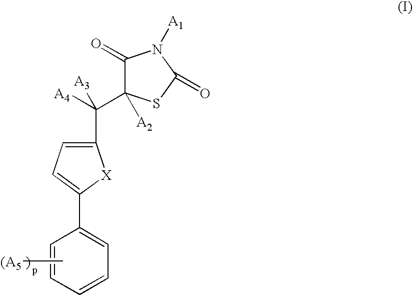
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

21. The regime or regimen as defined by claim 20, comprising maintaining said at least one compound of formula (I) or salt and/or solvate thereof in contact with said keratinous fibers and/or skin from whence said fibers emerge, and optionally rinsing same.

22. A regime or regimen for cosmetically treating the hair and/or the scalp of a human subject, comprising topically applying thereon a thus effective amount of at least one phenylfurylmethylthiazolidine-2,4-dione or phenylthienylmethylthiazolidine-2,4-dione compound having the following structural formula (I) or salt and/or solvate thereof: 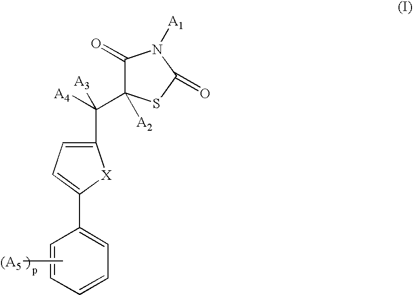
in which:
a) X is O or S;
b) A₁ is:
1) a hydrogen atom;
2) a linear or branched and saturated or unsaturated C₁-C₂₀ alkyl radical optionally substituted by one or more Z₁ groups;
c) the A₂, A₃, A₄ and A₅ radicals, which may be identical or different, are each:
1) a hydrogen atom;
2) a linear or branched and saturated or unsaturated C₁-C₂₀ alkyl radical optionally substituted by one or more Z₁ radicals;
3) a saturated or unsaturated hydrocarbon ring member having 3 to 7 atoms which is optionally substituted by one or more Z₁ groups;
4) a Z₁ radical;
d) p ranges from 0 to 5 inclusive and, in the event that p≧1, the A₅ substituents can be identical or different;
e) Z₁ is:
1) a halogen, such as F, Cl or Br;
2) a group selected from among CF₃, OCF₃, CN, NO₂, OZ₂, OCOZ₂, OCONZ₂Z′₂, SZ₂, SCOZ₂, SCONZ₂Z′₂, SCSOZ₂, SCSNZ₂Z′₂, NZ₂Z′₂, NZ₂C(═NZ′₂)NZ″₂Z′″₂, NZ₂SO₂Z′₂, COZ₂, COOZ₂, CONZ₂Z′₂, CSZ₂, CSNZ₂Z′₂, SO₃Z, SO₂NZ₂Z′₂, SO₂Z₂, SiZ₂Z′₂Z″₂ or Si(OZ₂)(OZ′₂)OZ″₂;
3) a linear or branched and saturated or unsaturated C₁-C₂₀ alkyl radical;
4) a saturated or unsaturated ring member having 4 to 15 atoms, optionally containing at least one heteroatom selected from among O, N or S, optionally fused to another ring, these rings optionally being substituted by one or more OZ₂, CF₃, halogen or linear or branched and saturated or unsaturated C₁-C₂₀ alkyl radicals, optionally containing a carbonyl or thiocarbonyl functional group; with the proviso that when A₅ is CH₂, Z₂ and Z′₂ are other than H and Me;
f) the Z₂, Z′₂, Z″₂ and Z′″₂ radicals independently are each:
1) a hydrogen atom;
2) a linear or branched and saturated or unsaturated C₁-C₂₀ alkyl radical optionally substituted by one or more Z₃ groups;
3) a saturated or unsaturated ring member having 4 to 15 atoms, optionally containing at least one heteroatom selected from among O, N or S, optionally fused to another ring, these rings optionally being substituted by one or more Z₃ groups;
g) the Z₃ radical is:
1) a Z₄ group;
2) a linear or branched and saturated or unsaturated C₁-C₂₀ alkyl radical optionally substituted by one or more Z₄ groups;
3) a saturated or unsaturated ring member having 4 to 15 atoms, optionally containing at least one heteroatom selected from among O, N or S, optionally fused to another ring, these rings optionally being substituted by one or more Z₄ groups;
h) Z₄ is:
1) a halogen, such as F, Cl or Br;
2) one of the groups CF₃, OCF₃, CN, NO₂, OZ₅, OCOZ₅, OCONZ₅Z′₅, SZ₅, SCOZ₅, SCONZ₅Z′₅, SCSOZ₅, SCSNZ₅Z′₅, NZ₅Z′₅, NZ₅COZ′₅, NZ₅CONZ′₅Z″₅, NZ₅C(═NZ′₅)NZ″₅Z′″₅, NZ₅SO₅Z′₅, COZ₅, COOZ₅, CONZ₅Z′₅, CSZ₅, CSNZ₅Z′₅, SO₃Z, SO₂NZ₅Z′₅, SO₂Z₅, SiZ₅Z′₅Z″₅ or Si(OZ₅)(OZ′₅)OZ″₅;
i) the Z₅, Z′₅, Z″₅ and Z′″₅ radicals independently are each:
1) a hydrogen atom;
2) a linear or branched and saturated or unsaturated C₁-C₂₀ alkyl radical;
3) a saturated or unsaturated ring member having 4 to 15 atoms, optionally containing at least one heteroatom selected from among O, N or S, optionally fused to another ring, these rings optionally being substituted by one or more CF₃, halogen or linear or branched and saturated or unsaturated C₁-C₂₀ alkyl radicals.

23. The regime or regimen as defined by claim 20, said human keratinous fibers comprising eyelashes and/or eyelids.
